(19) United States
(12) Patent Application Publication
(10) Pub. No.: US 2023/0346649 A1
(43) Pub. Date: Nov. 2, 2023

(54) SKIN MASK COMPOSITIONS AND METHODS OF USE

(71) Applicant: AQUAVIT PHARMACEUTICALS, INC., Las Vegas, NV (US)

(72) Inventor: Sobin Chang, Las Vegas, NV (US)

(21) Appl. No.: 18/025,790

(22) PCT Filed: Sep. 10, 2021
(86) PCT No.: PCT/US2021/049910

§ 371 Date: Mar. 10, 2023

Related U.S. Application Data
(60) Provisional application No. 63/076,630, filed on Sep. 10, 2020.

(51) Int. Cl. A61K 8/02; A61M 35/00; A61Q 19/08

(52) U.S. Cl. CPC A61K 8/0212; A61M 35/10; A61Q 19/08; A61M 2037/0023

(57) ABSTRACT

The invention provides skin mask compositions and methods of treating or preventing one or more skin conditions in a subject caused by extended use of PPE, comprising applying a skin mask composition to the face of the subject.

### CROSS REFERENCE TO RELATED APPLICATIONS

This application claims the benefit if U.S. Provisional Application No. 63/076,630, filed Sep. 10, 2020, the contents of which are incorporated by reference herein in their entirety.

### SUMMARY

It is to be understood that both the foregoing general description of the embodiments and the following detailed description are exemplary, and thus do not restrict the scope of the embodiments.

In one aspect, the invention provides skin mask compositions that can be applied to the face of a subject.

In another aspect, the invention provides a method of treating or preventing one or more skin conditions in a subject (such as, for example, one or more skin conditions caused by extended use of personal protective equipment (PPE) masks during the present COVID-19 pandemic), comprising applying a skin mask of the invention to the face of the subject.

In some embodiments, the skin mask is a gel-type skin mask comprising 0.3% to 5% alginate, alginic acid, sodium alginate and/or cellulose added with water.

In some embodiments, the skin mask is applied to the subject and covers the bottom portion of the layer of a subject's face, corresponding to a region of the face covered by a PPE mask. In some embodiments, the skin mask covers the bottom portion of the subject's face but does not cover the nose.

In some embodiments, the skin mask is applied to the subject and covers the upper portion of a subject's face, including the nose.

In some embodiments, the skin mask is for daytime use.

In some embodiments, the skin mask covers both the upper and lower portions of a subject's face.

In some embodiments, the skin mask is for nighttime use.

In some embodiments, the composition of the skin mask is targeted to control acne breakouts, hydration, cooling, or various combinations thereof.

In some embodiments, the skin mask comprises of a combination of ingredients selected from alginic acid, alginate, cellulose, agar, glycerin, niacinamide, carrageenan, dipropylene glycol, 1.2-hexanediol, hydroxyacetophenone, potassium chloride, butylene glycol, glucomannan, xanthan gum, Ceratonia siliqua gum, algin, allantoin, adenosine, Disodium EDTA, Laminaria japonica extract, dipotassium glycyrrhizate, salicylic acid, Eclipta prostrata leaf extract, Ulmus davidiana root extract, Amaranthus caudatus seed extract, dextrin, hydrogenated lecithin, sucrose, fructooligosaccharides, Ficus carica fruit extract, Centella asiatica extract, beta-glucan, Cetraria islandica extract, hydrolyzed hyaluronic acid, and ceramide NP.

In some embodiments, the skin mask ingredient compositions include, but are not limited to, 70% water, 22% glycerin, 2% niacinamide, 1% carrageenan, 1% dipropylene glycol, 1% 1,2-hexanediol, 0.5% hydroxyacetophenone, 0.3% potassium chloride, 0.2% butylene glycol, 0.1% glucomannan, 0.1% xanthan gum, 0.1% Ceratonia siliqua (carob) gum, 0.1% algin, 0.05% allantoin, 0.05% adenosine, 0.02% disodium EDTA, 0.01% Laminaria japonica extract, 0.01% dipotassium glycyrrhizate, 0.01% salicylic acid, 0.01% Eclipta prostrata leaf extract, 0.01% Ulmus davidiana root extract, 0.01% Amaranthus caudatus seed extract, up to 0.005% of dextrin, up to 0.005% hydrogenated lecithin, up to 0.005% sucrose, up to 0.005% fructooligosaccharides, up to 0.005% Ficus carica (fig) fruit extract, up to 0.005% Centella asiatica extract, up to 0.001% beta-glucan, up to 0.001% Cetraria islandica extract, up to 0.001% hydrolyzed hyaluronic acid, and up to 0.001% ceramide NP.

In some embodiments, the skin mask's main ingredient compositions include, but not limited to, 60 to 80% water, 20 to 25% glycerin, 1 to 5% niacinamide, 1 to 5% carrageenan, 0.1 to 2% hydroxyacetophenone, 0.5 to 2% algin, 0.1 to 2% allantoin, 0.1 to 2% adenosine, 0.005 to 0.05% salicylic acid, 0.01 to 1% disodium EDTA, 0.005 to 1% Laminaria japonica extract, 0.005 to 1% dipotassium glycyrrhizate, 0.005 to 1% Eclipta prostrata leaf extract, 0.005 to 1% Ulmus davidiana root extract, 0.005 to 1% Amaranthus caudatus seed extract, up to 1% Centella asiatica extract, up to 1% hydrolyzed hyaluronic acid, up to 1% Cetraria islandica extract, up to 1% ceramide NP.

In some embodiments, the skin mask's supplementary ingredient compositions include, but not limited to, 0.5 to 2% dipropylene glycol, 0.5 to 2% 1,2-hexanediol, 0.1 to 1% potassium chloride, 0.05 to 0.5% butylene glycol, 0.05 to 0.5% glucomannan, 0.05 to 0.5% xanthan gum, 0.05 to 0.5% Ceratonia siliqua (carob) gum, 0.001 to 1% of dextrin, 0.001 to 1% hydrogenated lecithin, 0.001 to 1% sucrose, 0.001 to 1% fructooligosaccharides, up to 1% Ficus carica (fig) fruit extract, up to 1% beta-glucan.

In some embodiments, the hydrogel mask is made by dissolving water soluble ingredients in water, and mixing with lipid soluble ingredients under set temperatures such as, for example, 60-90 degrees Celsius, to result in a homogenous transparent mixture of all compositions in the hydrogel, then, cooling the hydrogel in a set mold to create a facial skin mask.

In some embodiments, the daytime skin mask is worn under a PPE mask during routine, everyday activities.

In some embodiments, the application of the gel mask is used with a microneedle delivery device. In some embodiments, the microneedles only penetrate the stratum corneum and do not penetrate into the epidermal or dermal layers.

In some embodiments, the applications of the gel skin mask is used after the application of skin serums.

In some embodiments, a mobile software scans the face of the user and detects wrinkles and acne that allows the user to receive a skin mask personalized to fit the dimensions of the face area, skin type, etc.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a layout of the gel skin mask for daytime; covering only the bottom portion of a subject's face without covering the nasal bone.

FIG. 2 is a layout of the gel skin mask for daytime; covering only the bottom portion of a subject's face up to right below the nasal bone.

FIG. 3 is a layout of the gel skin mask for nighttime; covering the top and bottom portions of a subject's face separately.

FIG. 4 is a layout of the gel skin mask for nighttime; combined image of top and bottom.

FIG. 5 is an image of how a daytime skin gel mask, covering the lower half of the face shaded in blue, would be placed under a PPE face mask, margined in black.

FIG. 6 is an image of how a daytime skin gel mask, covering the nose bridge and lower half of the face shaded in blue, would be placed under a PPE face mask, margined in black.

FIG. 7 is an image of a microneedle infusion system.

FIG. 8 is a detailed image of the head of a microneedle infusion system.

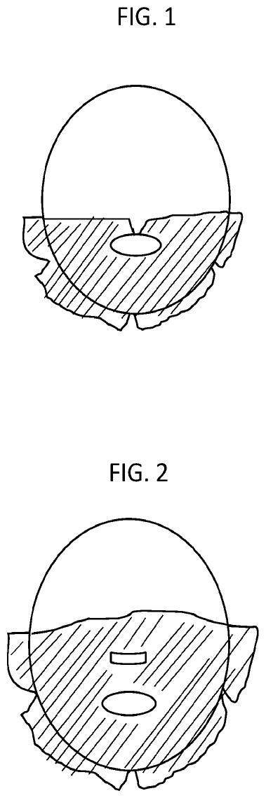

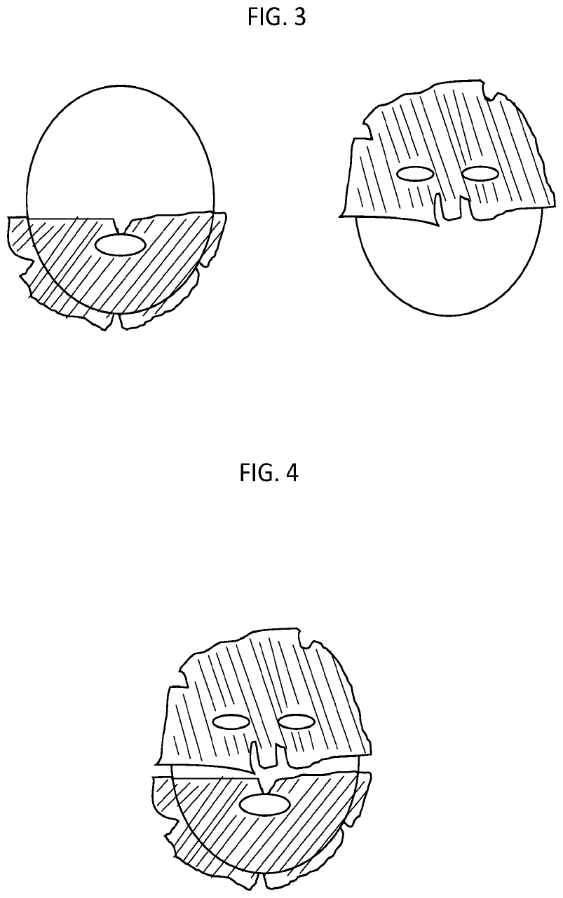

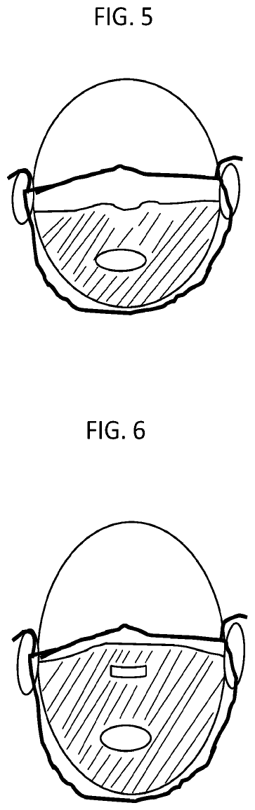

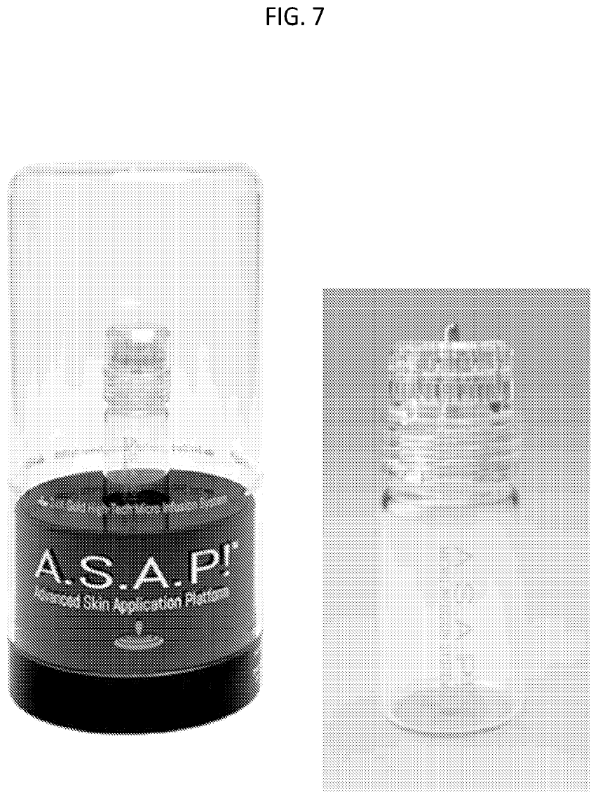

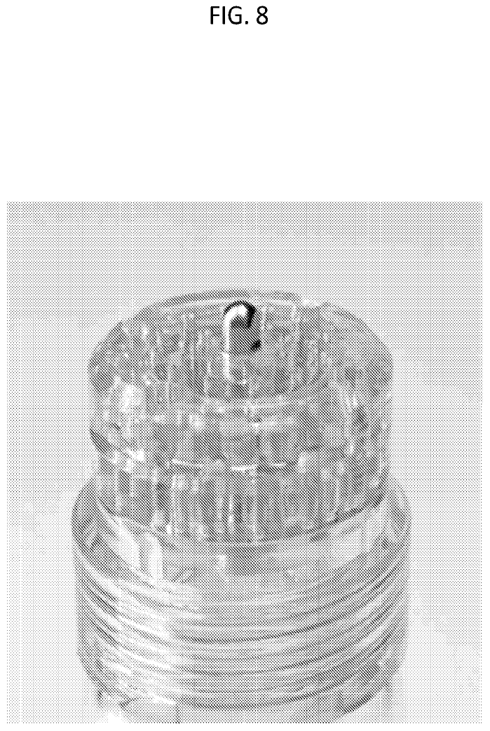

### DETAILED DESCRIPTION

Reference will now be made in detail to the presently preferred embodiments of the invention which, together with the drawings and the following examples, serve to explain the principles of the invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. The use of “skin mask”, “gel mask”, “sheet mask” are interchangeable and not intended to be limiting. The use of “personal protective equipment mask”, “PPE mask”, “face mask”, and/or “facial mask” are interchangeable and not intended to be limiting. Furthermore, where the description of one or more embodiments uses the term “containing,” those skilled in the art would understand that, in some specific instances, the embodiment or embodiments can be alternatively described using the language “consisting essentially of”, “composing of” and/or “consisting of.”

In some embodiments, the invention provides skin mask compositions that can be applied to the face of a subject.

In some embodiments, the invention provides a method of treating or preventing one or more skin conditions in a subject (such as, for example, one or more skin conditions caused by extended use of personal protective equipment (PPE) masks during the present COVID-19 pandemic), comprising applying a skin mask of the invention to the face of the subject.

In some embodiments, the skin mask comprises a gel.

In some embodiments, the skin masks are disposable after use

In some embodiments, the skin mask can be reused.

In some embodiments, the skin mask is composed to substantially cover only the bottom half of the face: lower cheek, perioral (chin, area under the nose), along the mandible. In another embodiment, a daytime skin mask is composed to cover substantially only the bottom half of the face, as stated above, including the up to right below the nasal bone. The skin mask use is not limiting. It can be used during the day, at night, in combination with other treatments, etc. In some embodiments, the skin mask is applied to the face during daytime use and the subject wears a PPE face mask over the skin mask.

In some embodiments, the skin mask is composed of top and bottom portions to substantially cover the entire face. In some embodiments, the top portion substantially covers the forehead, along the hairline, temple, periorbital area (underneath the eye, area between the temple and the eye, nose bridge), upper cheek, nose. In some embodiments, the bottom portion substantially covers the same parts as the daytime use skin mask. This could be used as, but not limited to, nighttime skin masks.

In some embodiments, the skin mask comprises one or more agents useful for the treatment of acne and/or skin blemish. In some embodiments, the one or more agents includes, but is not limited to salicylic acid, benzoyl peroxide, tea tree oil, glycolic acid, lactic acid, niacinamide having a concentration of (but not limited to) about 0.1%, about 0.2%, about 0.3%, about 0.4%, and up to about 0.49% and combinations thereof. In some embodiments, the skin mask can comprise one or more agents for hydration and/or moisturizing, including but not limited to hyaluronic acid, aloe vera, glycerin, and combinations thereof. In some embodiments, the skin mask can comprise one or more agents or cooling, including but not limited to menthol, peppermint extract and combinations thereof. In some embodiments, the skin mask can comprise one or more agents for skin tightening and anti-aging, including but not limited to collagen peptides, vitamin A, vitamin E, antioxidants, alpha hydroxy acids, including citric acid, glycolic acid, malic acid, tartaric acid, Y27632, apocynin and combinations thereof. In some embodiments, the skin mask comprises one or more additional agents, including but not limited to, humectants such as glycerin, thickening agents such as carbomer and cellulose, flavoring and exfoliating agents such as avocado extract, aloe vera extract, watermelon, mango, walnut extracts and preservatives such as paraben, benzoic acid, sodium benzoate and combinations thereof. In some embodiments, the skin mask comprises a single active agent while in other embodiments the skin mask comprises a combination of active agents. For example, in some embodiments, the skin mask comprises salicylic acid and hyaluronic acid to control acne and target hydration at the same time. In another example, the skin mask comprises lactic acid for blemish and menthol for effecting a cooling sensation.

In some embodiments, the skin mask can comprise one or more agents typically found in commercial serums that are applied to skin. In some embodiments, the skin mask comprises one or more growth factors such as epidermal growth factor (EGF), platelet-derived growth factor (PDGF), and polydeoxyribonucleotides (PDRN). In some embodiments, the skin mask may comprise niacinamide and tranexamic acid. In some embodiments, the skin mask comprises polyglutamic acid and adenosine. In some embodiments, the skin masks can comprise one or more additional agents including but not limited to hyaluronic acid, distilled water, Centella asiatic extract, Polygonum cuspidatum root extract, Rosmarinus officinalis, glycerin, Scutellaria baicalensis root extract, Chamomilla recutita (matricaria) flower extract, Glycyrrhiza glabra (licorice) root extract, Camellia sinensis leaf extract, etc., and combinations thereof. In some embodiments, the serum skin masks may be individualized but also combined with each other to meet the users' needs.

In some embodiments, the daytime skin mask is worn under a PPE mask. Due to the COVID-19 pandemic, wearing a personal protective face mask has become routine and in some jurisdictions required. The extended wearing of PPE face masks and other PPE such as gloves, face shields, and protective clothes has increased acne outbreaks, oil production, blemish, itching, scarring, desquamation, etc., particularly among healthcare workers. In some embodiments, the daytime skin mask will soothe/control the users' needs with the different types of skin masks that can be worn under the face mask. In some embodiments, the user applies the skin mask before placing on their face a PPE face mask.

The duration of use of the skin mask is not limiting. In some embodiments, the skin mask can be worn for about 1 minute, about 5 minutes, about 10 minutes, about 15 minutes, about 20 minutes, about 25 minutes, about 30 minutes or longer. This can allow the user to continue one's daily activities while being able to treat or prevent any condition affecting their skin, e.g. conditions that arise from wearing a PPE face mask. For example, in some embodiments, a user can apply the skin mask in the morning and wear the face mask during a commute to work, and then remove the skin mask after, e.g., after about 30 mins, and continue one's day with their skin care already finished. The skin care routine is not limited to conditions or damage done only from a PPE face mask, but can include any conditions affecting the skin, for example, conditions caused by outdoor activities, sunlight exposure, sweat, makeup, etc.

In some embodiments, the application of the gel skin mask is performed using a microneedle device. In some embodiments, the microneedle device comprises one or more microneedles that do not penetrate into the epidermal or dermal skin layers but only disrupt the stratum corneum layer of skin. In some embodiments, the gel or serum or solution is delivered by a microneedle device through twenty (20) 0.2 mm microneedles that penetrate the skin and apply the solution directly into the skin where the damage is or where supplementation is needed. With a gentle repeated up and down motion of the head onto the skin to apply the solution, eventually the solution forms a skin mask onto the user's face. Due to this personalized applicator, the skin mask can be used on the user's face, neck, arm, shoulders, legs, or anywhere the user wishes.

In some embodiments, the skin mask is applied after administering one or more serums with a microneedle device to enhance the outcome or treat or prevent a skin condition. In some embodiments, the skin mask is highly recommended to be used together with one or more serums to enhance the outcome associated with the administration. For example, in some embodiments, one can use the microneedle device to administer skin serums. In some embodiments, the skin serums may contain one or more of the following ingredients: Water, Dipropylene Glycol, Niacinamide, Betaine, Sodium Hyaluronate, Butylene Glycol, Centella asiatica Extract, Disodium EDTA, Allantoin, Alcohol Denat., Polygonum cuspidatum Root Extract, Scutellaria baicalensis Root Extract, Aureobasidium pullulans Ferment Filtrate, Camellia sinensis Leaf Extract, Glycyrrhiza glabra (Licorice) Root Extract, Chamomilla recutita (matricaria) Flower Extract, Rosmarinus officinalis (Rosemary) Leaf Extract, Glutathione, Tranexamic Acid, Zinc PCA, Chlorella vulgaris/Lupinus albus Protein Ferment, Nasturtium Officinale Flower/Leaf extract, Glycerin, Sodium DNA, Stem Cell, Taurine, Acanthopanax senticosus Root Extract, Sodium Benzoate, Potassium Sorbate, Pentylene Glycol, Copper Tripeptide-1, Adenosine, PEG-40 Hydrogenated Castor Oil, Streptococcus thermophilus/Cyclopia intermedia Leaf/Stem Extract/Lactose/Whole Dry Milk Ferment, PPG-26-Buteth-26, 1,2-Hexanediol, Caprylyl Glycol, Polyglutamic Acid, Acetyl Hexapeptide-8, Palmitoyl Tripeptide-1, Tripeptide-1, Hexapeptide-9, Caprylic/Capric Triglyceride, Palmitoyl Tripeptide-5, Lecithin, 7-Dehydrocholesteryl Palmitate. After the first application, one will apply the skin mask to capture the ingredients onto the skin even more. In some embodiments, a subject can use different types of skin masks based on the serums applied on the skin. In some embodiments, the skin mask compositions can be packaged in a kit. In some embodiments, the kit comprises a plurality of samples (e.g., 30 skin mask compositions). In some embodiments, the kit further comprises one or more serums, and one or more microneedle delivery devices. In some embodiments, the kit further comprises one or more information sheets comprising instructions for using the compositions and devices. In some embodiments, the user can purchase a subscription and be supplied the kit on a periodic (e.g., monthly) basis.

In some embodiments, the invention further provides means for scanning the face of the user. In some embodiments, the means comprises a mobile software or application that could be utilized on a portable electronic device such as a smartphone. In some embodiments the software or application detects wrinkles and acne which allows the user to receive a skin mask personalized to meet one's needs. In some embodiments, the software or application scans the facial dimensions of the user, analyzes the skin type and prepares a skin mask personalized for the user. Personalization features can include, but are not limited to the unique skin mask size fitting to the user's face, ingredient mixtures as the user demands, aroma/flavor/scent, etc.

1. A method of treating or preventing one or more skin conditions in a subject caused by extended use of PPE, comprising applying a skin mask composition to the face of the subject, wherein the skin mask is selected from the group consisting of:
a. a 2 piece gel type skin mask, separated laterally, top and bottom, which allows separate applications;
b. a daytime skin mask covering the bottom half of the face, and worn under the PPE mask;
c. a skin mask covering the lower half of the face, not including the nose;
d. a skin mask covering the lower half of the face, including up to right below the nasal bone; and
e. a nighttime skin mask worn on the full face.

2. The method of claim 1, wherein the nighttime skin mask treats one or more conditions caused by PPE that is other than a mask, including PPE that is selected from face shields or goggles.

3. The method of claim 1, wherein the skin mask composition comprises ingredients for soothing, calming, controlling blemishes and acne.

4. The method of claim 1, wherein the composition comprises ingredients for hydrating, and moisturizing the skin.

5. The method of claim 1, wherein the skin mask is used after using a microinfusion system with different skin serums.

6. The method of claim 1, wherein the application of the skin mask is performed by a microneedle delivery device.

7. The method of claim 1, wherein the skin mask is personalized for each user through a mobile application scanning the facial features such as wrinkles, fine lines, acne blemish, scars, etc.
